(19) United States
(12) Patent Application Publication
(10) Pub. No.: US 2004/0161363 A1
(43) Pub. Date: Aug. 19, 2004

(54) Apparatus and method for thermal sterilization of liquids

(76) Inventor: Wilhelm Lutzer, Zarpen (DE)

Correspondence Address: FASSE PATENT ATTORNEYS, P.A., P.O. BOX 726, HAMPDEN, ME, 04444-0726, US

(21) Appl. No.: 10/757,764

(22) Filed: Jan. 14, 2004

(30) Foreign Application Priority Data
Jan. 16, 2003 (DE) 103 01 376.8

(51) Int. Cl. A61L 2/00; A61L 9/00

(52) U.S. Cl. 422/38; 422/1; 422/307

(57) ABSTRACT

A heater and a counterflow heat exchanger are arranged inside a pressure vessel, having an inlet connected to a pressurizing device and an outlet connected to a depressurizing device. To thermally sterilize a microbiologically contaminated liquid, the inflowing contaminated liquid is pressurized and pumped into the pressure vessel, where it is pre-heated by flowing along the heat exchanger and then heated to the required treatment temperature by the heater. The arising treated liquid is cooled by flowing along the heat exchanger and is then depressurized upon exiting the pressure vessel. Thermal energy is transferred from the outflowing liquid to the inflowing liquid by the heat exchanger. Pressure energy is transferred from the outflowing liquid to the inflowing liquid by the interconnected depressurizing and pressurizing devices. A high proportion of the total required energy is retained within the system, and high treatment temperatures (above the atmospheric boiling point) are efficiently achievable.

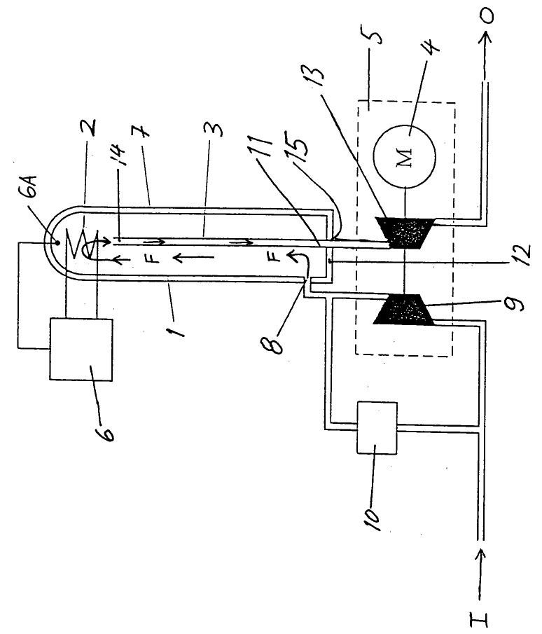

### PRIORITY CLAIM

This application is based on and claims the priority under 35 U.S.C. §119 of German Patent Application 103 01 376.8, filed on Jan. 16, 2003, the entire disclosure of which is incorporated herein by reference.

### FIELD OF THE INVENTION

The invention relates to an apparatus and a method for thermally sterilizing or pasteurizing a liquid and especially water, for destroying microbiological contaminants, by flowing the liquid through a heating zone in which the water is heated to a sufficient treatment temperature for a sufficient treatment dwell time to destroy the target microbiological contaminants or microorganisms.

### BACKGROUND INFORMATION

It is generally known to sterilize various liquids, such as drinking water and liquid foodstuffs by exposing the liquid to a sufficiently high temperature for a sufficiently long thermal treatment dwell time to destroy at least the desired high percentage of the target microbial contaminants or microorganisms. Such thermal sterilization is also know as pasteurization. In this context, it is known that different microbial contaminants or microorganisms can survive for different lengths of time at different temperatures, so that different thermal treatment dwell times at the elevated temperature will be required for different treatment temperatures and/or for different microorganisms. It is further known that the required thermal treatment dwell time, during which the liquid must be maintained at the prescribed treatment temperature, generally decreases as the temperature increases. This is true for treatment apparatuses that carry out a batch treatment process as well as treatment apparatuses that carry out a flow-through process, i.e. in which the thermal sterilization treatment is to be achieved with an essentially continuous flow of the liquid through the apparatus.

Problems arise in the conventional methods and apparatuses for achieving a thermal sterilization, due to a thermal inefficiency, which leads to an undesirable high energy consumption and/or makes it impossible to achieve sufficiently high sterilization temperatures for a sufficiently long dwell time to destroy the most temperature-resistant microbial contaminants or microorganisms.

### SUMMARY OF THE INVENTION

In view of the above, it is an object of the invention to provide an apparatus and a method for thermally sterilizing or pasteurizing liquids, by achieving treatment temperatures lying above the normal atmospheric boiling temperature of the relevant liquid. Another object of the invention is to improve the thermal and operating efficiency of such an apparatus and method, to achieve improved economy and also allow higher treatment temperatures to be achieved and maintained for a sufficiently long treatment dwell time. The invention further aims to avoid or overcome the disadvantages of the prior art, and to achieve additional advantages, as apparent from the present specification. The attainment of these objects is, however, not a required limitation of the present invention.

The above objects have been achieved according to the invention in an apparatus for the thermal sterilization of liquids, including a pressure vessel with an inlet and an outlet for the liquid to be thermally sterilized, a heater arranged in a heating zone within the pressure vessel, and a counterflow heat exchanger arranged in the pressure vessel by means of which the warmer out-flowing treated liquid transfers heat to the cooler in-flowing contaminated liquid.

The above objects have further been achieved according to the invention in a method of thermally sterilizing a liquid, including pressurizing the liquid to an elevated pressure above normal atmospheric pressure, flowing the pressurized liquid along a heat exchanger to pre-heat the liquid, flowing the pre-heated liquid through a heating zone where the liquid is heated to a sufficiently high sterilizing treatment temperature, flowing the treated liquid along the heat exchanger to transfer heat from the treated liquid via the heat exchanger to the inflowing liquid, whereby the treated liquid is cooled and the inflowing liquid is pre-heated, and then reducing the pressure of the out-flowing treated liquid.

According to a preferred feature of the invention, a substantial portion (e. g. a predominant proportion greater than 50% or even greater than 75%) of the heating energy required for heating the in-flowing liquid to the required thermal treatment temperature is provided by heat transfer from the out-flowing treated liquid in the counterflow heat exchanger. Thereby, the out-flowing liquid is cooled while the in-flowing liquid is pre-heated, and the substantial portion of the total required heating energy is retained within the pressure vessel. This improves the thermal efficiency of the process in the operation of the apparatus, so that the thermal sterilization becomes more economical, and also allows a higher treatment temperature to be achieved more quickly and maintained for a sufficient treatment dwell time.

According to further preferred features of the invention, a pressure increasing device such as a pressurizing pump is connected on the inlet side of the pressure vessel, while a pressure reducing device such as a pressure reducing turbine or expansion device is connected on the outlet side of the pressure vessel. The term “pump” is intended to have broad coverage of all devices that convey and pressurize a liquid. The pressure reducing device may have a similar construction as the pump, but operating in an opposite flow direction. Preferably, these two devices are connected to each other, for example through a suitable mechanical transmission or shaft, so that the energy recovered by the pressure reducing device while reducing the pressure of the pressurized liquid is provided to drive the pressure increasing device for pressurizing the liquid on its way into the pressure vessel. A motor may be additionally connected to the pressure increasing device to provide additional required drive power, for example to make up for mechanical power losses. In this manner, the pressurization cycle is also very efficient and economical. Only the minimal thermal and pressure energy losses must be added to the system, for example in the form of electrical energy supplied to the heater and to the pump motor.

The most important advantage of the invention is that the in-flowing liquid can be brought to a very high temperature at which only a very short treatment dwell time is necessary. The treatment temperature is not limited to the atmospheric boiling point of the liquid being treated, due to the high pressurization. For example, in this context the critical case relates to the high temperature resistant bacterium “Bacillus stearthermophilus”, which exhibits a decimal reduction time that is approximately halved for every 10 K increase of the treatment temperature above 130° C. A so called D10-dwell time (i.e. a reduction of the bacteria to 10% of the original population number) at 200° C. can be determined to be 0.0875 seconds through extrapolation from published data. For a reduction to 1/100 or 1% of the original number of bacteria, which is presumably the intended target reduction, it is thus necessary to provide a treatment dwell time of 0.875 seconds at a temperature of 200° C. The term “sterilization” as used herein thus does not require 100% sterility, but merely a desired degree of reduction of the microbiological contamination.

This required dwell time at the required temperature of at least 200° C. is advantageously achievable in a flow-through process with the apparatus and method according to the invention. This is especially achieved because of the combination of the high pressure and the pre-heating by heat exchange that is achieved in the pressure vessel in the inventive apparatus. Namely, the required temperature of 200° C. is achieved at a high pressure of approximately 16 bar in the pressure vessel of the inventive apparatus. Operating at this high pressure allows the required high temperatures above the normal atmospheric boiling point of the liquid to be achieved, while still preferably maintaining the liquid in its liquid state throughout the treatment process.

### BRIEF DESCRIPTION OF THE DRAWINGS

In order that the invention may be clearly understood, it will now be described in connection with an example embodiment with reference to the accompanying single drawing FIGURE, which schematically shows an apparatus for the thermal sterilization of liquids, preferably water, according to the invention.

### DETAILED DESCRIPTION OF A PREFERRED EXAMPLE EMBODIMENT AND THE BEST MODE OF THE INVENTION

The exemplary apparatus according to the invention shown schematically in the single drawing FIGURE comprises a pressure vessel 1, a regulatable heater 2, preferably an electric resistance heater 2, arranged in a heating zone within the pressure vessel 1, a counterflow heat exchanger 3 arranged within the pressure vessel 1, and a pressurizing-depressurizing module or compression-expansion module 5. A regulator 6 is connected to the heater 2 and receives a temperature signal from a temperature sensor 6A arranged in the heating zone within the pressure vessel 1. Thereby, the regulator 6 applies e.g. electrical energy to the heater 2 in a regulated manner dependent on the temperature signal provided by the temperature sensor 6A, so as to regulate the operation of the heater 2 to achieve the required treatment temperature in the heating zone. The pressure vessel 1 is preferably surrounded and enclosed by any suitable conventionally known thermal insulation 1, and preferably has an elongated cylindrical shape to provide a sufficient effective length for heat exchange through the heat exchanger 3 as will be described below.

The pressure vessel 1 has an inlet 8 and an outlet 15, through which the liquid to be treated, preferably water, enters and exits from the pressure vessel 1. In the illustrated preferred embodiment, the heat exchanger 3 is embodied as a simple pipe extending along the central axis of the cylindrical pressure vessel 1, and is connected to or extends through the outlet 15 in the center of the flat bottom end wall or floor 12 of the pressure vessel 1. Thus, the outlet end 11 of the heat exchanger 3 connects to or penetrates outwardly through the outlet 15, while the inlet end 14 of the heat exchanger 3 is open in the pressure space within the interior of the pressure vessel 1 at or proximate to the heating zone around the heater 2. Alternatively, the inlet of the heat exchanger can be connected to the vessel inlet 8 while the heat exchanger outlet is simply open in the pressure space within the vessel proximate to the heating zone.

In the illustrated example embodiment, the microbiologically, e.g. bacterially, contaminated in-flowing water flows into the system at 1 and then into the interior space of the pressure vessel 1 through the inlet 8, to flow at F along the outside of the pipe-shaped heat exchanger 3 to be pre-heated, then into and through the heating zone defined by the heater 2 to heat the water to the required treatment temperature and form thereof treated water. The treated water then flows into the inlet end 14 of the heat exchanger 3, and through the interior of the heat exchanger 3 while it gives up heat, and then leaves the pressure vessel 1 through the outlet 15 via the heat exchanger outlet 11, whereupon the out-flowing water then exits the system as an outflow at O. Thereby, the cooler in-flowing water entering the pressure vessel 1 at the inlet 8 is pre-heated by receiving heat from the out-flowing water that flows through the inside of the heat exchanger 3. This heat exchange through the pipe wall of the heat exchanger 3 simultaneously serves to cool the out-flowing treated water while pre-heating the in-flowing contaminated water. Thereby, a substantial portion of the energy required for heating the in-flowing liquid to the treatment temperature is extracted from the out-flowing treated liquid through the heat exchanger 3, and thereby remains within the pressure vessel 1. The heater 2 must only provide the additional heating energy to make up for heat losses through the insulation 7 and due to incomplete re-cooling and heat transfer from the out-flowing liquid through the heat exchanger 3.

The simple embodiment schematically illustrated in the FIGURE uses a straight pipe-shaped heat exchanger 3 with a pipe wall that physically separates yet thermally couples the in-flowing contaminated water and the out-flowing treated water on opposite sides (outside and inside) of the pipe wall. In this regard, the pipe wall may be made of any suitable metal, for example. Other heat exchanger designs are possible. A heat exchanger can alternatively comprise plural straight pipes or a spiral configuration pipe, or the like, to achieve a more compact arrangement or to increase the heat exchange surface area as required. Additionally or alternatively, the pipe of the heat exchanger 3 could be fitted with fins or ribs to increase the heat exchange area. As a further alternative, as mentioned above, the heat exchange pipe could be connected to the inlet 8 rather than the outlet 11, so that the in-flowing cool contaminated water flows through the inside of the heat exchanger 3, while the out-flowing hot treated water flows along the outside of the heat exchanger. Another alternative embodiment of the heat exchanger involves simply a dividing wall that divides the pressure space within the pressure vessel into two or more longitudinal flow passages on opposite sides of the dividing wall.

The pressurizing-depressurizing or compression-expansion module 5 serves to pump the water through the pressure vessel 1 at the appropriate rate to achieve the required treatment dwell time in the heating zone, while also pressurizing the interior of the pressure vessel 1 to the required high pressure, for example approximately 16 bar. At this high pressure, the water will be maintained in its liquid state even when it is heated to 200° C. in the heating zone.

The pressurizing-depressurizing module 5 includes a pressure increasing device such as a pressurizing pump 9 having an input that receives the inflow 1 of contaminated water and an output. connected to the inlet 8 of the pressure vessel 1. Thereby, the in-flowing contaminated water is pressurized to the required high pressure and caused to flow into the interior space of the pressure vessel 1 and along the outside of the heat exchanger 3. Furthermore, an excess pressure relief such as a pressure relief valve 10 is connected in parallel to the pressurizing pump 9 between the input and the output thereof to avoid excessive pressurization of the pressure vessel 1. The module 5 further includes a pressure reducing device such as a pressure reducing or expansion turbine 13 having an input connected to the outlet 11 of the heat exchanger 3, e.g. to the outlet 15 of the vessel 1, and an output that provides the outflow O of cooled, de-pressurized, thermally sterilized water.

In this manner, the pressure increasing device 9 is connected in series upstream of the inlet 14 of the heat exchanger 3 and in fact upstream of the inlet 8 of the pressure vessel 1, while the pressure reducing device 13 is connected in series downstream of the outlet 11 of the heat exchanger 3 and the outlet 15 of the pressure vessel 1. This allows the pressure reducing device 13 to extract substantial pressurization energy from the water as it is depressurized, and to return that energy through a mechanical transmission or shaft, for example, to the pressure increasing device 9, for pressurizing the new water being pumped into the pressure vessel 1. Extra required pumping and pressurizing energy, e.g. to make up for mechanical losses and the like, is provided by an electric motor 4 or the like, which is also connected to the drive shaft of the pressure increasing device 9.

A preferred application or installation of the method and apparatus according to the invention is for the sterilization of water, e.g. purified wastewater or graywater, to produce potable water therefrom, on board an aircraft. Nonetheless, more generally, this method and apparatus can further be used in any application or installation requiring the preparation of sterilized potable water or the pasteurization of liquid foodstuffs.

Although the invention has been described with reference to specific example embodiments, it will be appreciated that it is intended to cover all modifications and equivalents within the scope of the appended claims. It should also be understood that the present disclosure includes all possible combinations of any individual features recited in any of the appended claims.

What is claimed is:

1. An apparatus for thermal sterilization of a microbiologically contaminated liquid to produce a thermally treated liquid, said apparatus comprising:
a pressure vessel enclosing a pressure space therein and having a vessel inlet and a vessel outlet;
a heater arranged in a heating zone in said pressure space within said pressure vessel; and
a heat exchanger arranged in said pressure vessel, having a heat exchanger inlet, a heat exchanger outlet and a heat exchange wall defining first and second flow paths along opposite sides of said heat exchange wall;
wherein at least one of said heat exchanger inlet and outlet is connected to at least one of said vessel inlet and outlet respectively, so that the contaminated liquid can flow into said pressure vessel through said vessel inlet, along said first flow path in a first net flow direction, and through said heating zone to from the treated liquid, and the treated liquid can flow from said heating zone along said second flow path in a second net flow direction and out of said pressure vessel through said vessel outlet, while heat is transferred from the treated liquid to the contaminated liquid through said heat exchange wall.

2. The apparatus according to claim 1, wherein said heat exchanger is a counterflow heat exchanger, and the second net flow direction is opposite the first net flow direction.

3. The apparatus according to claim 2, wherein said heat exchanger is configured, dimensioned, arranged and adapted so that a substantial proportion of energy needed for heating the contaminated liquid is retained inside said pressure vessel by re-cooling of the treated liquid along said second flow path of said heat exchanger before the treated liquid exits said pressure vessel.

4. The apparatus according to claim 1, wherein only one of said heat exchanger inlet and outlet is connected to only one of said vessel inlet and outlet respectively at a distal end of said heat exchanger relative to said heating zone, and another of said heat exchanger inlet and outlet is open within said pressure space in said pressure vessel at a proximal end of said heat exchanger relative to said heating zone.

5. The apparatus according to claim 1, wherein said heat exchanger outlet is connected to said vessel outlet at a distal end of said heat exchanger relative to said heating zone, and said heat exchanger inlet is open within said pressure space in said pressure vessel at a proximal end of said heat exchanger relative to said heating zone.

6. The apparatus according to claim 1, wherein said second flow path is bounded and enclosed by said heat exchange wall and thereby confined inside said heat exchanger, and said first flow path is a portion of said pressure space surrounding said heat exchanger within said pressure vessel.

7. The apparatus according to claim 1, wherein said heat exchanger has a hollow pipe-shape, and said heat exchange wall is a cylindrical pipe wall enclosing said second flow path therein.

8. The apparatus according to claim 1, wherein said heater is an electric resistance heater, and further comprising a temperature sensor arranged in said heating zone within said pressure vessel, and a power regulator connected to said temperature sensor to receive a temperature signal therefrom and connected to said electric resistance heater to apply thereto electrical power regulated dependent on the temperature signal.

9. The apparatus according to claim 1, further comprising a pressurizing pump outside of said pressure vessel connected to said vessel inlet.

10. The apparatus according to claim 9, further comprising a pressure-reducing device outside of said pressure vessel connected to said vessel outlet.

11. The apparatus according to claim 10, wherein said pressure-reducing device is connected for energy transmission to said pressurizing pump.

12. The apparatus according to claim 11, comprising a common shaft or a mechanical transmission by which said pressure-reducing device is connected to said pressurizing pump.

13. The apparatus according to claim 11, wherein said pressurizing pump and said pressure-reducing device together form a pressurizing-depressurizing module.

14. The apparatus according to claim 11, further comprising a drive motor connected to said pressurizing pump.

15. The apparatus according to claim 9, further comprising an excess pressure relief device connected to said vessel inlet parallel to said pressurizing pump.

16. The apparatus according to claim 1, further comprising a pressure-reducing turbine outside of said pressure vessel connected to said vessel outlet.

17. An apparatus for thermal sterilization of a microbiologically contaminated liquid, said apparatus comprising:
a pressure vessel enclosing a pressure space therein and having a vessel inlet and a vessel outlet;
a heater arranged in a heating zone in said pressure space within said pressure vessel;
a counterflow heat exchanger arranged in said pressure vessel, having a proximal end and a distal end relative to said heating zone, said proximal end being open to said pressure space within said pressure vessel, and said distal end being connected for flow communication with said vessel inlet or said vessel outlet; and
a pressurizing pump outside of said pressure vessel connected to said vessel inlet.

18. The apparatus according to claim 17, further comprising a pressure-reducing device outside of said pressure vessel connected for flow communication with said vessel outlet, and connected mechanically for power transmission to said pressurizing pump.

19. A method for thermally sterilizing a microbiologically contaminated liquid to produce a thermally treated liquid, comprising the steps:
a) pressurizing said contaminated liquid;
b) flowing said contaminated liquid in a first flow direction along a first side of a heat exchanger thereby pre-heating said contaminated liquid with heat transferred from said heat exchanger;
c) flowing said contaminated liquid through a heating zone, and in said heating zone, heating said contaminated liquid with a heater to a sufficiently high treatment temperature for achieving the thermal sterilization of said contaminated liquid to produce said treated liquid;
d) flowing said treated liquid in a second flow direction opposite said first flow direction along a second side of said heat exchanger physically separated from and in thermal communication with said contaminated liquid on said first side of said heat exchanger, thereby cooling said treated liquid by transferring heat from said treated liquid to said heat exchanger; and
e) reducing a pressure of said treated liquid.

20. The method according to claim 19, wherein said step e) further comprises recovering energy from said reducing of said pressure, and then further comprising using said energy in performing said step a).

21. The method according to claim 19, wherein said treatment temperature in said step c) is above a normal atmospheric boiling point of said contaminated liquid, and said contaminated liquid is maintained in a liquid state because said contaminated liquid is pressurized to above atmospheric pressure in said step a).

22. The method according to claim 19, wherein said contaminated liquid is pressurized to a pressure of at least 16 bar in said step a) and said pressure is maintained in said steps b), c) and d), and wherein said treatment temperature in said step c) is at least 200° C.
